(19) United States
(12) Patent Application Publication
(10) Pub. No.: US 2026/0088169 A1
(43) Pub. Date: Mar. 26, 2026

(54) SYSTEM AND METHOD FOR DETECTION OF DISEASE

(71) Applicant: Intus Biosciences, LLC, Farmington, CT (US)

(72) Inventors: Mark Driscoll, Wallingford, CT (US); Daniel Fasulo, Madison, CT (US)

(21) Appl. No.: 18/445,677

(22) Filed: Sep. 23, 2024

(51) Int. Cl. G16H 50/20; G16B 30/00; G16B 40/20; G16H 10/40; G16H 50/30; G16H 50/70

(52) U.S. Cl. CPC G16H 50/20; G16B 30/00; G16B 40/20; G16H 10/40; G16H 50/30; G16H 50/70

(57) ABSTRACT

The present invention provides systems and methods for the detection of systemic disease from bacterial content samples obtained from a subject. Also provided are methods of training a machine learning algorithm to correlate patient microbiome sequence data with a disease state or disease development risk. These systems and methods utilize a high-resolution, database-independent, high-throughput microbial profiling platform to diagnose systemic disease in patients or to identify those patients at risk of developing systemic disease. Also provided are systems and kits for carrying out the methods.

### TECHNICAL FIELD

The present invention provides systems and methods for the detection of systemic disease from bacterial content samples obtained from a subject. Also provided are methods for training a machine learning (“ML”) algorithm to correlate patient microbiome sequence data with a disease state or the risk of developing a disease state. These systems and methods utilize a high-resolution, database-independent, high-throughput microbial profiling platform to diagnose systemic disease in patients or to identify those patients at risk of developing systemic disease. Also provided are kits for carrying out the methods.

### BACKGROUND

There is an ongoing need to develop safe, reliable, and noninvasive systems and methods for detecting disease in a subject or for determining risk factors for or the propensity to develop the disease.

A prime example of this diagnostic need is in the medical area of colorectal cancer (“CRC”). As reported by the Centers for Disease Control (CDC)., in 2019, 142,462 cases of colon and rectum cancer were reported, corresponding to an incidence rate of 36 per 100,000 standard population. Especially concerning is the trend of the increasing incidence of CRC amongst those under 50 years of age. Similarly, there is a need for earlier and more accurate diagnostic methods for a wide range of disease states and indications including neurodegenerative diseases, Alzheimer's Disease, Parkinson's Disease, Amyotrophic Lateral Sclerosis (ALS), Multiple Sclerosis (MS), Lewy Body Dementia, Frontotemporal Dementia, Spinocerebellar Ataxia, autoimmune diseases, Celiac Disease, Crohn's Disease, Ulcerative Colitis, Inflammatory Bowel Disease (IBD), Rheumatoid Arthritis, Type 1 Diabetes, Hashimoto's Thyroiditis, Graves' Disease, Psoriasis, Sjögren's Syndrome, Systemic Lupus Erythematosus (SLE), Myasthenia Gravis, Vasculitis, Pemphigus Vulgaris, Dermatomyositis, Guillain-Barré Syndrome, digestive disorders, Diverticulitis, Pancreatitis, Irritable Bowel Syndrome (IBS), Gastroesophageal Reflux Disease (GERD), Peptic Ulcer Disease, Non-Alcoholic Fatty Liver Disease (NAFLD), metabolic disorders, Type 2 Diabetes, Obesity, Hyperthyroidism, Hypothyroidism, cardiovascular diseases, Coronary Artery Disease, Hypertension (High Blood Pressure), Congestive Heart Failure, Stroke, Atherosclerosis, Renal (Kidney) disease, Chronic Kidney Disease (CKD), Polycystic Kidney Disease, Nephrotic Syndrome, Cancer, Lung Cancer, Breast Cancer, Prostate Cancer, Colon Cancer, Colorectal cancer, Early Onset Colorectal Cancer, Leukemia, Lymphoma, Pancreatic Cancer, Ovarian Cancer, Melanoma, Bladder Cancer, Liver Cancer, Kidney (renal cell and renal pelvis) Cancer, mental health disorders, Depression, Anxiety Disorders, Bipolar Disorder, Schizophrenia, Obsessive-Compulsive Disorder (OCD), Post-Traumatic Stress Disorder (PTSD), substance use disorders, Alcohol Use Disorder, Opioid Use Disorder, Nicotine Dependence, Chronic Obstructive Pulmonary Disease (COPD), Asthma, Fibromyalgia, Gout, Osteoarthritis, and Osteoporosis.

In the past identification and quantification of microbes has been used as a direct measure of their presence and amount to identify the cause and severity of diseases, such as bacterial, fungal, or viral infections.

There is a need to improve the sensitivity of non-invasive tests to detect diseases or risk of diseases, particularly where the early detection is important, such as, for example with colorectal and breast cancer.

Metagenomics and shotgun sequencing has been used in the past to identify and determine the relative prevalence of pathogenic bacteria from biological samples, such as gut microbiome samples from human patients. However, metagenomic and shotgun sequencing methods will not work efficiently and have limitations such as requiring a very large and impractical number of sequencing runs.

However, there is a more significant need to develop platforms and methods not for the direct detection of the microorganisms per se, but rather to determine and develop methods of microbiota analysis for screening and detection methods for disease, such as chronic or system disease. There have been microbiological methods in place to directly determine the presence of, e.g., a bacterial infection, since the early days of microbiology and microscopy in the later 1800s. However, there is a lack of platforms and methods to take advantage of and utilize the connection between microbial presence and the diagnosis of diseases, or the propensity to develop a disease.

Current technologies rely on identifying the DNA of the microbes of concern. The present invention does not rely on this. Instead, the present invention identifies a piece of the DNA, utilizing an amplicon, which is a piece of DNA (or RNA), which is distinctive for or identifies the microbe. In other words, the present invention does not need to identifying full DNA sequences, but rather small DNA pieces. Because of this difference, it is not necessary to identify the underlying microbes per se. The power of the present invention is based on the methods it utilizes for training a machine learning algorithm to correlate patient microbiome sequence data with a disease state, where this is done without the need to identify the underlying microbes. The present invention is providing the diagnostics using subtle, patterns of small DNA sequences, i.e. the presence, lack, or relative amounts, of these sequences in the sample as a whole, as indicators of the disease state or propensity or risk for contracting the disease state.

What the present invention provides is distinguished from directly identifying and quantifying microbes (e.g., bacteria) to determine an infection of the microbes. Instead, the present invention does not need to determine the presence and relative quantity of the microbes that may be present as a direct measure of a disease (e.g., an infection), but instead determines the presence and quantity of the microbes as a proxy for, or marker for, as a diagnostic method for identifying a systemic disease state, or the propensity for developing that disease state. In other words, the present invention is not interested in identifying the pathogenic microbes for the sake of the microbes themselves, but goes beyond to determine what their presence and/or quantity means as a determinant or predictor of a disease state, e.g., a systemic or chronic disease state. Additionally, rather than taxonomical classification, unique genetic features in microbiome bacteria are utilized which requires no prior knowledge of bacterial taxonomy. That is, the present invention does not associate taxonomical bacterial composition with a disease state, but rather accurately predicts a disease state based upon unique bacterial genetic feature associations with the disease state.

The present invention goes beyond the limitations of methods for directly determining a disease state or condition from the types and quantities of pathogenic microorganisms. Instead, the present platforms and methods provide bridge the gap to detecting or determining the risk for develop. The present invention goes beyond the limits of current diagnostic technologies. Therefore, the present invention provides powerful methods for the early detection of chronic and systemic disease states or the risk of developing these disease states.

### SUMMARY

The present invention provides systems and methods for the detection of systemic disease from bacterial content samples obtained from a subject. Also provided are methods for training a machine learning algorithm to correlate patient microbiome sequence data with a disease state. What is significant is that these systems and methods utilize a high-resolution, database-independent, high-throughput microbial profiling platform to diagnose systemic disease in patients or to identify those patients at risk of developing systemic disease. Also provided are kits for carrying out the methods for the detection of systemic disease.

The present invention includes, but is not limited to, various aspects of the below numbered embodiments. In various further embodiments, these aspects may be combined with each other and/or with various aspects of the present disclosure.
1. A method for diagnosing or predicting the development of a disease state from microbiome sequence data from a prospective patient, comprising the following steps:
   a. collecting biological specimens and metadata from a patient cohort having the disease state and from a patient cohort lacking the disease state;
   b. generating microbiome sequence data from the biological specimens;
   c. processing the microbiome sequence data to generate features having a quantified relevance to the disease state for each patient;
   d. associating metadata with the generated features for each patient;
   e. selecting a subset of the features to generate a reduced feature set;
   f. training a machine learning algorithm on the reduced feature set to create a classification model that classifies the patient status as having the disease state, an early form of the disease state, lacking the disease state, or being at risk for developing the disease state;
   g. obtaining microbiome sequence data and metadata from a prospective patient;
   h. quantifying the features in the reduced feature set from the microbiome sequence data of the prospective patient; and
   i. applying the classification model to the quantified features in the reduced feature set from the prospective patient to determine whether the prospective patient has or lacks the disease state, or are is risk for developing the disease state.
2. The method of embodiment 1, wherein the features can be matched and compared across patients.
3. The method of embodiment 2, further comprising applying data transformations to calibrate, normalize, or quantize the features for comparison across patients.
4. The method of embodiment 1, where the microbiome sequence data is the 16S rRNA gene and flanking upstream and downstream genomic regions, in part or in whole.
5. The method of embodiment 1, where the microbiome sequence data begins in or upstream of the 16S rRNA gene and extends past the end of the 16S rRNA gene as a contiguous amplicon sequence.
6. The method of embodiment 1, wherein the microbiome sequence data comprises one or more of 16S, ITS, and 23S sequences.
7. The method of embodiment 6, wherein the microbiome sequence data comprises the 16S-ITS-23S amplicon.
8. The method of embodiment 1, where the quantified relevance to the disease state of each feature is determined by (i) grouping the reads across samples into Operational Taxonomic Units (OTUs) or Amplicon Sequence Variants (ASVs),
   (ii) creating a representative sequence for each OTU or ASV, and
   (iii) counting the number of reads matching each OTU or ASV representative in each sample.
9. The method of embodiment 1, where the quantified relevance to the disease state of each feature is defined to be the number of occurrences of the feature in the microbiome sequence data.
10. The method of embodiment 1, wherein the length of each feature is approximately 5 to 100 nucleotides.
11. The method of embodiment 1 in which the biological specimens are fecal samples, blood samples, CSF samples, urine samples, saliva samples, other internal or external bodily fluids, skin swabs, gum swabs, vaginal swabs, or swabs of specific internal or external anatomical features.
12. The method of embodiment 1, further comprising:
   obtaining fecal immunochemical test data from one or more of the patient cohort having the disease state and the patient cohort lacking the disease state; and
   training the machine learning algorithm on the reduced feature set and the fecal immunochemical test data to create the classification model.
13. The method of embodiment 12, further comprising:
obtaining fecal immunochemical test data from the prospective patient; and applying the classification model to the quantified features in the reduced feature set from the prospective patient and the fecal immunochemical test data from the prospective patient to determine whether the prospective patient has or lack the disease state, or are is risk for developing the disease state.
14. The method of embodiment 1, in which the disease state is a neurodegenerative disease, Alzheimer's Disease, Parkinson's Disease, Amyotrophic Lateral Sclerosis (ALS), Multiple Sclerosis (MS), Lewy Body Dementia, Frontotemporal Dementia, Spinocerebellar Ataxia, autoimmune disease, Celiac Disease, Crohn's Disease, Ulcerative Colitis, Inflammatory Bowel Disease (IBD), Rheumatoid Arthritis, Type 1 Diabetes, Hashimoto's Thyroiditis, Graves'Disease, Psoriasis, Sjögren's Syndrome, Systemic Lupus Erythematosus (SLE), Myasthenia Gravis, Vasculitis, Pemphigus Vulgaris, Dermatomyositis, Guillain-Barré Syndrome, digestive disorder, Diverticulitis, Pancreatitis, Irritable Bowel Syndrome (IBS), Gastroesophageal Reflux Disease (GERD), Peptic Ulcer Disease, Non-Alcoholic Fatty Liver Disease (NAFLD), metabolic disorders, Type 2 Diabetes, Obesity, Hyperthyroidism, Hypothyroidism, cardiovascular disease, Coronary Artery Disease, Hypertension (High Blood Pressure), Congestive Heart Failure, Stroke, Atherosclerosis, Renal (Kidney) disease, Chronic Kidney Disease (CKD), Polycystic Kidney Disease, Nephrotic Syndrome, Cancer, Lung Cancer, Breast Cancer, Prostate Cancer, Colon Cancer, Colorectal Cancer, Early Onset Colorectal Cancer, Leukemia, Lymphoma, Pancreatic Cancer, Ovarian Cancer, Melanoma, Bladder Cancer, Liver Cancer, Kidney (renal cell and renal pelvis) Cancer, mental health disorder, Depression, Anxiety Disorders, Bipolar Disorder, Schizophrenia, Obsessive-Compulsive Disorder (OCD), Post-Traumatic Stress Disorder (PTSD), substance use disorder, Alcohol Use Disorder, Opioid Use Disorder, Nicotine Dependence, Chronic Obstructive Pulmonary Disease (COPD), Asthma, Fibromyalgia, Gout, Osteoarthritis, Osteoporosisa cancer, a neurodegenerative disorder, digestive disorder, colorectal cancer, early-onset colorectal cancer, pancreatic cancer, breast cancer, prostate cancer, lung cancer, bladder cancer, liver cancer, kidney (renal cell and renal pelvis) cancer, Alzheimer's disease, Parkinson's disease, ALS, celiac disease, Crohn's disease, ulcerative colitis, diverticulitis, pancreatitis, Type Diabetes, Type 2 Diabetes, Rheumatoid Arthritis, IBD, IBS, Cardiovascular Disease, Non-alcoholic Fatty Liver Disease, Chronic Kidney Disease, Alcohol Use Disorder, and Mental Health Disorder.
15. A method of training a machine learning algorithm to correlate patient microbiome sequence data with a disease state:
   obtaining sequence data for a first plurality of patients having a diagnosed disease state and for a second plurality of control patients lacking the disease state, wherein the sequence data of the first and second pluralities of patients comprises respective computer-readable microbiome nucleotide sequences from biological samples collected from the respective patients;
   identifying sequence features from the microbiome nucleotide sequences which correlate positively or negatively with the disease state;
   generating machine learning training data comprising:
      i) at least a subset of the identified sequence features,
      ii) for each of the identified sequence features, their property of corellating positively or negatively with the disease state, and
      iii) retrospective patient data comprising computer-readable microbiome nucleotide sequences from biological samples collected from a plurality of retrospective patients having the disease state and/or a plurality of retrospective patients lacking the disease state; and
   training the machine learning algorithm with the machine learning training data to predict the presence or absence of the disease state in the retrospective patient data.
16. The method of embodiment 15, wherein training the machine learning algorithm produces a model capable of predicting the presence or absence of the disease state in prospective patients having no known disease state.
17. The method of embodiment 15, wherein the method includes no taxonomic identification of bacterial strains in the microbiome nucleotide sequences.
18. The method of embodiment 15, wherein the microbiome nucleotide sequences comprise bacterial nucleotide sequences.
19. The method of embodiment 18, wherein the bacterial nucleotide sequences comprise one or more of 16S, ITS, and 23S sequences.
20. The method of embodiment 19, wherein the bacterial nucleotode sequences comprise the 16S-ITS-23S amplicon.
21. The method of embodiment 20, wherein the training data includes no taxonomic identification bacterial strains from the 16S-ITS-23S amplicons.
22. The method of embodiment 15, wherein the sequence data and retrospective patient data are proportional to the bacterial populations in the underlying biological samples.
23. The method of embodiment 15, wherein the machine learning training data further comprises retrospective patient data comprising computer-readable microbiome nucleotide sequences from biological samples collected from a plurality of retrospective patients lacking the disease state.
24. The method of embodiment 15, in which the disease state is a neurodegenerative disease, Alzheimer's Disease, Parkinson's Disease, Amyotrophic Lateral Sclerosis (ALS), Multiple Sclerosis (MS), Lewy Body Dementia, Frontotemporal Dementia, Spinocerebellar Ataxia, autoimmune disease, Celiac Disease, Crohn's Disease, Ulcerative Colitis, Inflammatory Bowel Disease (IBD), Rheumatoid Arthritis, Type 1 Diabetes, Hashimoto's Thyroiditis, Graves' Disease, Psoriasis, Sjögren's Syndrome, Systemic Lupus Erythematosus (SLE), Myasthenia Gravis, Vasculitis, Pemphigus Vulgaris, Dermatomyositis, Guillain-Barré Syndrome, digestive disorder, Diverticulitis, Pancreatitis, Irritable Bowel Syndrome (IBS), Gastroesophageal Reflux Disease (GERD), Peptic Ulcer Disease, Non-Alcoholic Fatty Liver Disease (NAFLD), metabolic disorders, Type 2 Diabetes, Obesity, Hyperthyroidism, Hypothyroidism, cardiovascular disease, Coronary Artery Disease, Hypertension (High Blood Pressure), Congestive Heart Failure, Stroke, Atherosclerosis, Renal (Kidney) disease, Chronic Kidney Disease (CKD), Polycystic Kidney Disease, Nephrotic Syndrome, Cancer, Lung Cancer, Breast Cancer, Prostate Cancer, Colon Cancer, Colorectal Cancer, Early Onset Colorectal Cancer, Leukemia, Lymphoma, Pancreatic Cancer, Ovarian Cancer, Melanoma, Bladder Cancer, Liver Cancer, Kidney (renal cell and renal pelvis) Cancer, mental health disorder, Depression, Anxiety Disorders, Bipolar Disorder, Schizophrenia, Obsessive-Compulsive Disorder (OCD), Post-Traumatic Stress Disorder (PTSD), substance use disorder, Alcohol Use Disorder, Opioid Use Disorder, Nicotine Dependence, Chronic Obstructive Pulmonary Disease (COPD), Asthma, Fibromyalgia, Gout, Osteoarthritis, Osteoporosis
25. The method of embodiment 15, wherein the machine learning training data further comprises:
   iv) fecal immunochemistry test collected from at least one of the plurality of retrospective patients having or lacking the disease state.
26. The method of embodiment 15, wherein the machine learning training data further comprises metadata from at least one of the plurality of retrospective patients having or lacking the disease state.
27. A system comprising;
   a computing device operable to execute computer-readable instructions, the computer-readable instructions being configured to perform the steps of:
      obtaining sequence data for a first plurality of patients having a diagnosed disease state and for a second plurality of control patients lacking the disease state, wherein the sequence data of the first and second pluralities of patients comprises respective computer-readable microbiome nucleotide sequences from biological samples collected from the respective patients;
      identifying sequence features from the microbiome nucleotide sequences which correlate positively or negatively with the disease state ;
      generating machine learning training data comprising:
         i) at least a subset of the identified sequence features,
         ii) for each of the identified sequence features, their property of corellating positively or negatively with the disease state, and
         iii) retrospective patient data comprising computer-readable microbiome nucleotide sequences from biological samples collected from a plurality of retrospective patients having the disease state and/or a plurality of retrospective patients lacking the disease state; and
      training a machine learning algorithm with the machine learning training data to predict the presence or absence of the disease state in the retrospective patient data.
28. A kit for diagnosing or predicting the development of a disease state from microbiome sequence data from a prospective patient, comprising:
a sample collector for obtaining biological specimens from a prospective patient and instructions for obtaining the biological specimens; wherein the collected biological specimens are useful for one or more of:
   a. generating microbiome sequence data from the biological specimens;
   b. processing the microbiome sequence data to generate features having a quantified relevance to the disease state for each patient;
   c. associating metadata with the generated features for each patient;
   d. selecting a subset of the features to generate a reduced feature set;
   e. training a machine learning algorithm on the reduced feature set to create a classification model that classifies the patient status as having the disease state, lacking the disease state, or being at risk for developing the disease state;
   f. obtaining microbiome sequence data and metadata from a prospective patient;
   g. quantifying the features in the reduced feature set from the microbiome sequence data of the prospective patient; and
   h. applying the classification model to the quantified features in the reduced feature set from the prospective patient to determine whether the prospective patients has or lacks the disease state, or are is risk for developing the disease state.

### BRIEF DESCRIPTION OF THE DRAWINGS

FIGS. 1A and 1B show the addition of training sample data yields continuous increases in sensitivity and specificity on 100 blinded samples. FIG. 1A shows the percent correct assignment of 100 blinded samples (y-axis), consisting of an unknown number of cancer (“CA”), advanced adenoma (“AA”), and normal controls. Samples were assigned to groups using classifiers determined by our machine learning algorithm after training on four training datasets: (i) ‘First Dataset’, (ii) ‘More Controls’, (iii) ‘More Controls and CA’, and (iv) ‘+FIT (fecal immunochemical test) data’. Training of our machine learning algorithms on the ‘First Dataset’ used fecal samples from 35 colorectal cancer, 35 advanced adenoma, and 178 non-cancer age-similar controls. In the ‘More Controls’ training dataset, identification of blinded samples was re-assessed after additional training using 43 additional control samples. After further ML training on 22 cancer and 35 additional controls in the ‘More Controls and CA’, sensitivity and specificity were again re-assessed. Finally, FIT data for each sample was added in the final analysis (+FIT). FIG. 1B shows the increasing percentages of correct calls of the 100 blind samples after each machine learning training as compared to the results obtained by a currently marketed diagnostic product.

FIGS. 2A, 2B, 2C, 2D, 2E, and 2F show the location and frequency of colorectal cancer screen features. The starting point of each feature is shown on the X-axis, the number of features observed is shown on the Y-axis. The features were either positively or negatively correlated with colorectal cancer, advanced adenoma or normal samples. The starting point was measured from the start of primer 27f in the 16S rRNA gene. The 23S rRNA gene is the final 600 bases on the right of the plot. Features in the Internally Transcribed Spacer (ITS) were plotted across 600 bases, although the ITS length varies. FIG. 2A shows the frequency of each feature in the 16S-ITS-23S rRNA amplicon, (i.e., the Titan-1™ Amplicon). FIG. 2B shows the common primer positions used in 16S studies are shown in red approximately to scale. FIG. 2C shows the 16S gene is represented by a striped arrow, with lighter variable regions and darker conservative regions, shown approximately to scale. The Internally ITS region between the 16S and 23S genes is shown in the center between the 16S and 23S regions, scaled to 600 bases, although many are shorter. The amplicon includes the first ˜600 bases of the 23S rRNA gene, represented by the rightmost arrow. FIG. 2D shows the 16S rRNA CRC feature count for the conserved and variable regions. The start and end position of each region is listed at left, counting from the start of the 27f primer. Columns detail the number of features, region size, and feature density. The 5 highest feature densities are all highlighted. FIG. 2E shows a comparison of 16S rRNA conserved or variable total features, region size, and feature density. FIG. 2F shows comparison of features, region size, and feature density of the 16S, ITS, and 23S regions of the amplicon.

FIGS. 3A, 3B, 3C, and 3D show CRC-specific sequence features contribute strong positive and negative correlations with CRC. The figures, which are in the form of bar graphs, show a small subset of bacterial sequence features detected as part of the ML/AI algorithm for CRC screening sort with CRC and non-CRC (Normal). The y-axis counts the number of samples, the colors indicate the sample type. The sequence feature abundance is shown on the x-axis, using a log2 scale. Samples that did not have the signal are shown at ‘0’ Feature Abundance. The taxonomy/name is summarizes the best taxonomy available for that feature after mapping the NCBI database. FIG. 3A shows data for fusobacterium, FIG. 3B shows data for an uncultured bacterium partial 16S rRNA gene, FIG. 3C shows data for an oral bacterium (labeled as Oral Bacterium 2), and FIG. 3D shows data for a gut bacterial pathogen (labeled as Gut Bacterial Pathogen 1).

FIG. 4. Shows that the bacterial profile is stable over 14 Months. Fecal samples from a single adult male, age 56 at the first time point, were sampled every few months over a 14 month period. Species above 1% relative abundance are shown. There was no significant change in health status, no antibiotic use, and no major change in diet over the 14-month period.

FIG. 5. Shows a projection reaching 96% or greater sensitivity and specificity. An exponential curve was used to estimate that training the ML algorithm on 50 additional CA, 65 AA, and 200 control samples will achieve 96% or better sensitivity and specificity for CRC and AA. Existing commercial technology on the 100 blinded sample set provides a 76% sensitivity and 82% specificity. The current performance on these same samples is 72% sensitivity and 90% specificity. The present invention with additional samples will provide a projected performance of 95% sensitivity and 96% specificity.

### DETAILED DESCRIPTION

There is strong evidence that human-associated bacteria can be drivers of chronic disease, including multiple cancers, autoimmune diseases, inflammatory gut disorders, metabolic disease, and diabetes. To explore putative associations between chronic disease and bacterial content, a platform controlling all steps from bacterial lysis through analysis was developed. The platform's substrain-level, high-throughput bacterial profiling data were optimized for a database-independent custom machine learning (ML) algorithm, useful for hypothesis-free discovery of chronic disease/bacterial relationships. Colorectal cancer (CRC) was selected as the first area of exploration for the platform, because over a decade of evidence correlates the presence of oral bacteria such as Fusobacterium nucleatum with biofilm-related colon tissue invasion and chronic inflammation resulting in CRC. The machine ML was trained on sequence data from well characterized reference CRC, advanced adenoma (AA), and healthy control fecal samples. CRC and AA-associated sequence features discovered de novo by the ML included the recently described CRC-associated Fusobacterium nuceatum animalis clade 2, Escherichia coli strains, as well as other known CRC-biofilm related oral and gut bacteria. The platform made independent discoveries of known CRC-associated bacteria using orders of magnitude less data and time than required for whole genome sequencing methods. The ML also discovered poorly described bacterial strains that are common in the population, but negatively associated with CRC. To test utility of the platform for CRC diagnosis, the most robust CRC-related bacterial sequence features discovered by the ML were combined into classifiers, which were used to characterize 100 blinded fecal samples, with accuracies comparable to currently available testing product results. Uniquely, as additional CRC and controls were added to the ML training dataset, CA/AA sensitivity and specificity for the blinded samples improved. Projections of performance based on the current ˜300 sample training set indicates that significant future improvement for both AA and CA is achievable. In addition, these results suggest that early detection of bacteria involved in biofilm formation may have utility as a novel CRC diagnosis and prevention method, identifying the initiation and progression of CRC-related biofilms potentially years before CRC can be detected using human ‘omics’. Routine fecal sample monitoring combined with non-invasive methods targeting the establishment and expansion of AA-and CRC-associated biofilms could be an effective CRC prevention tool.

We developed a high-resolution, database independent, high throughput fecal microbial profiling platform to generate novel insights into the relationship between bacteria and chronic disease. Our company, Intus Biosciences, now has validated performance of our platform, a non-invasive, high accuracy, high throughput platform that delivers strain-level bacterial profiling, using thousands of times less sequencing than current shotgun bacterial profiling methods. This platform enables the large amount of genomic data collection based on amplicon technology for utilization in the present invention to provides systems and methods for the detection of systemic disease from bacterial content samples obtained from a subject. This data is then used as the basis for providing methods for training a machine learning algorithm to correlate patient microbiome sequence data with a disease state.

Multiple chronic human diseases have been linked to gut bacterial content, for example, there has long been evidence that oral bacterial biofilm formation in the colon contributes significantly to the development and progression of CRC. Bacterial involvement is currently being studied in breast, pancreatic, and other cancers. Individual bacteria from humans with CRC, including strains of E. coli, Fusobacterium, Bacteroides fragilis, are also associated with increased colon tumor formation in animal models, but recent work indicates that complex microbial consortia may be required for inception of tumorigenisis.

CRC was selected as proof of principle that the data and analysis tools in the newly developed platform were both necessary and sufficient for finding associations between bacterial content and chronic disease. Methods of bacterial profiling to accurately detect AA and CRC have been explored for more than a decade, but lack of success indicates that practical technology to realize this goal has been unavailable. One key advantage of the platform is that because it is trained de novo to identify relationships between bacterial DNA sequence features and disease, the platform should independently re-discover bacteria that are known to be associated with AA and CRC, as well as identify novel sequence features from bacteria with undiscovered associations with CRC.

We detail the use of our platform for CRC and AA screening using bacterial DNA sequence features.

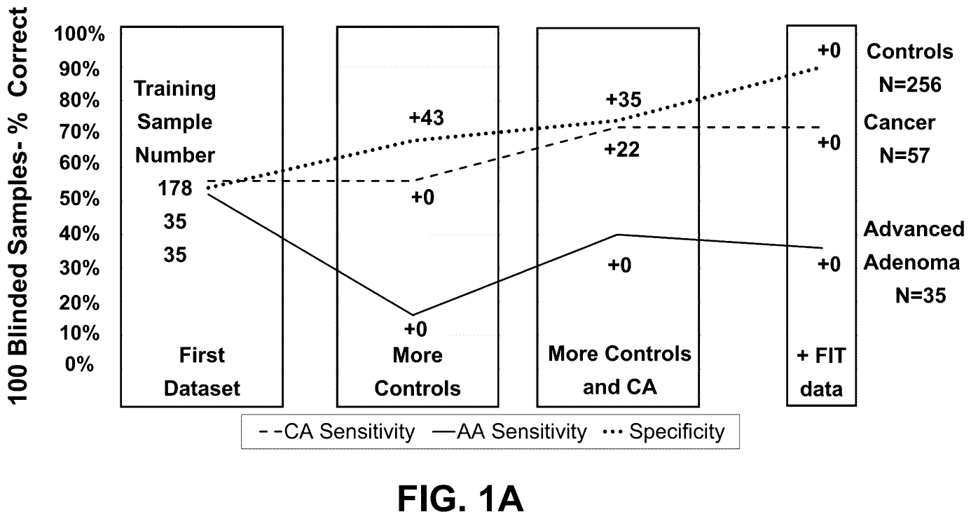

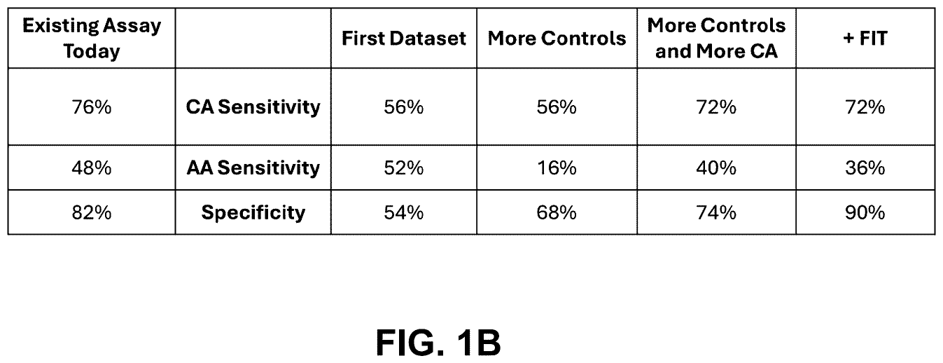

Our laboratory developed test (LDT) platform was used to profile well-characterized CRC, AA, and control fecal samples. Samples were prepared and sequenced using our platform lysis and multiple amplicon amplicon sequencing variants (ASV) bacterial strain fingerprint patterns as described previously2. Sequence data were analyzed using machine learning algorithm (ML)-based feature selection for each of four ML training datasets. The training datasets started with 35 CRC, 35 AA, and 178 control samples. In subsequent ML training, more control samples were added, more CRC samples were added, and finally, FIT data was added to the training datasets, to track the rate of improvement as the ML accessed more samples, to ascertain approximately how many samples would be needed to determine what constitutes bacterial profiles corresponding to CRC, AA and normal fecal samples. In each dataset, robust features (as described in materials and methods) identified across multiple samples in the training dataset were selected and combined using ML into classifiers for identifying AA and CA-positive samples. The classifiers resulting from the ML training datasets were used to screen 100 blinded samples, results of which were not shared with us prior. FIG. 1 details the continuous increases in sensitivity and specificity for CRC screening for the 100 blinded CRC/AA/Control samples as more CA positive, control samples, and corresponding FIT data were added to the training dataset.

Database independence is a requirement for CRC screening. Uniquely, the platform of the present invention is database independent, and therefore does not require taxonomic assignment of bacterial sequences for feature identification, combination of features into classifiers, or subsequent analysis/diagnosis of new samples. Avoiding taxonomic assignments is key to the accuracy of the platform. Taxonomy reliance presents issues that can obscure high resolution data and prevent successful CRC diagnosis, because important distinctions between bacteria required to find association with CRC are frequently not assigned differential taxonomies. In addition, taxonomies can change over time, taxonomies can be incorrectly assigned, and old assignments can get ‘more incorrect’ over time. Most importantly, most bacteria are not represented in any database, with no assigned taxonomy. We show how database independence is a basic function of our platform, a result of the combination of the 16S-ITS-23S bacterial amplicon, sequencing the amplicon in a single read, identification of single read amplicon sequence variants that are 100% correct representatives of the original genome, and, because bacterial genomes typically have multiple copies of the amplicon, a unique combination of amplicons generates a bacterial ASV amplicon fingerprint unique to that strain. Database independence is a major advantage because analysis of CRC data is free from the intrinsic errors, limitations, bias, and omissions of all analysis pipelines relying on the taxonomies of sequenced bacteria.

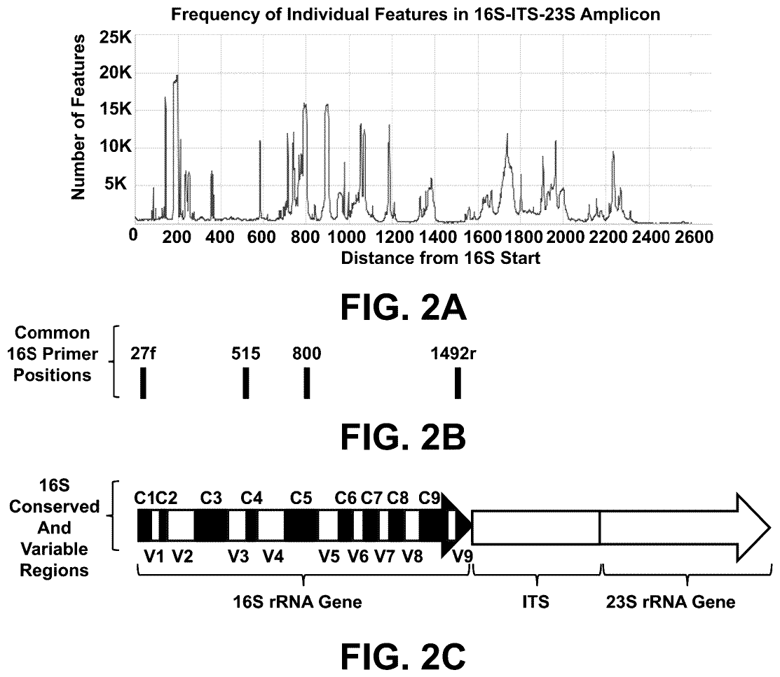

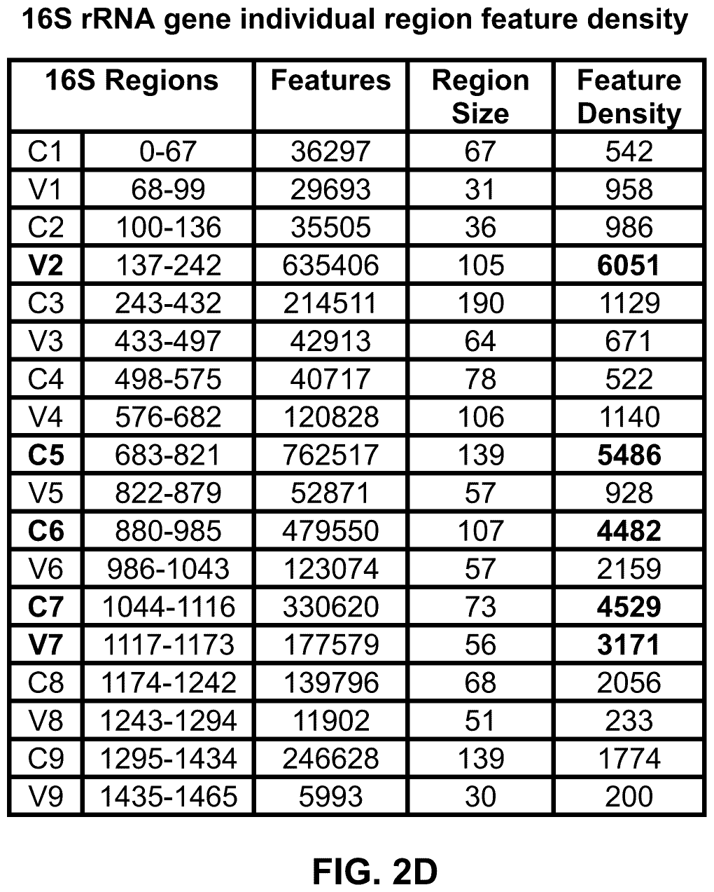

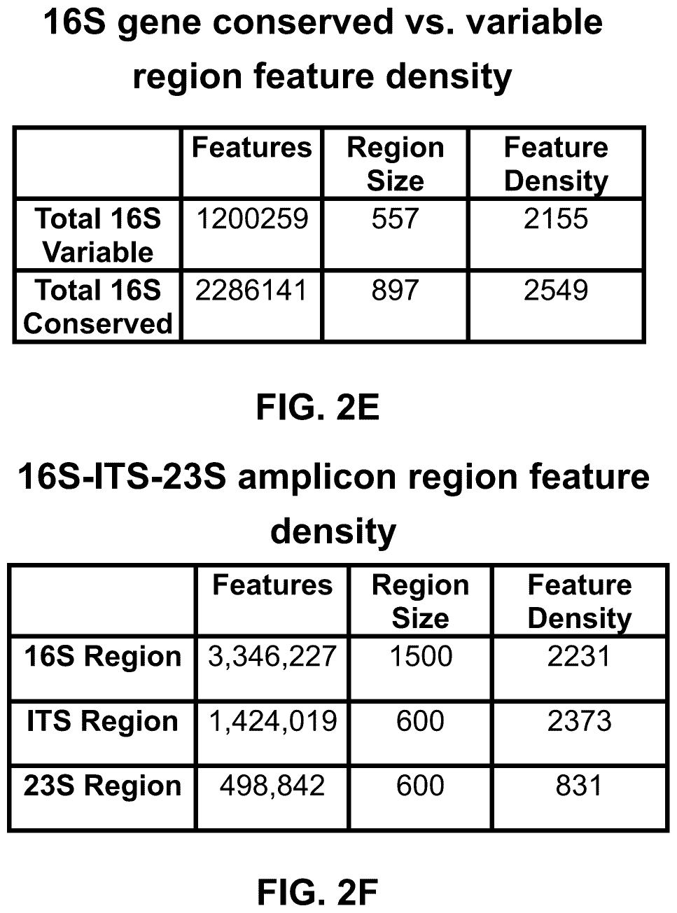

Signals for CRC/AA biofilm detection span the entire 16S-ITS-23S amplicon. The ML identified and combined sequence features that differentiated AA and CA-positive fecal samples to build database independent classifiers for screening the 100 blinded, unknown samples. Investigating the sequence features and feature combinations in the classifiers can provide context for why the ML is able to identify CRC-and AA-related bacterial biofilms associated with lesions, and may provide insight into CRC-related disease mechanisms. To explore the ML-generated classifiers, sequence features identified by the ML were plotted by starting location in FIG. 2A. Within the 16S and 23S regions of our amplicon there were relatively long stretches of sequence with few features, with intermittent spikes of highly discriminant features concentrated in specific regions. FIG. 2B depicts the location of four commonly used amplicon primer sites bracketing known 16S rRNA gene variable sites that are commonly used as amplicon targets for identifying bacteria, so different commonly used amplicons can be easily correlated with feature density in the Figure. FIG. 2C shows the locations of 16S rRNA gene conserved and variable regions, shown approximately to scale, as well as the ITS (Internally Transcribed Spacer) and the first ˜600 bases of the 23S gene that is included in our amplicon. The ITS is highly variable in size and sequence content within and across bacterial genomes. FIG. 2D details CRC and AA discriminant feature density within the 16S gene regions. The 16S V2 region has the highest density, but the more conserved regions C5, C6, and C7 had higher density than any of the other 8 variable regions. Overall, as seen in FIG. 2E, the total feature density is 16% higher in the conserved regions than in the variable regions. Unsurprisingly, FIG. 2F shows the highly variable ITS region from ˜1600 to ˜2000 had the highest feature density as compared to the 16S and the partial 23S gene within the amplicon.

Recent evidence demonstrates that strain-level resolution is a minimum requirement for accurate CRC screening; the ability to differentiate substrains/clades of Fusobacterium more likely to be associated with CRC is a primary example. Since the Fusobacterium nucleatum anamalis clade2 (Fna C2)/CRC clade-level association is well-studied and very specific, we investigated whether our database-independent amplicon sequence variation was sufficient for Fna C2 identification. A detailed analysis of the 90 CRC-related screening classifiers used in the automated CRC/AA screen revealed a total of 5 features that detected all 118 Fusobacterium isolates sequenced and published in Zepeda-Rivera et al. Importantly, there was one feature, designation ‘aaca74’, that selectively detected Fna C2, demonstrating that our ML independently discovered the CRC-Fna C2 association, which is integrated into the classifiers used to screen samples.

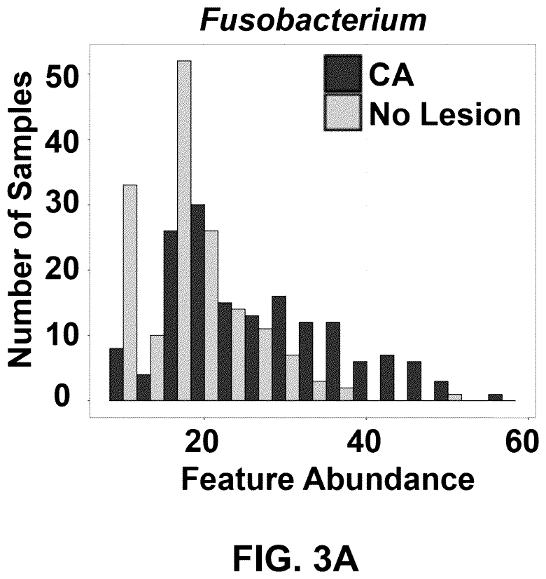

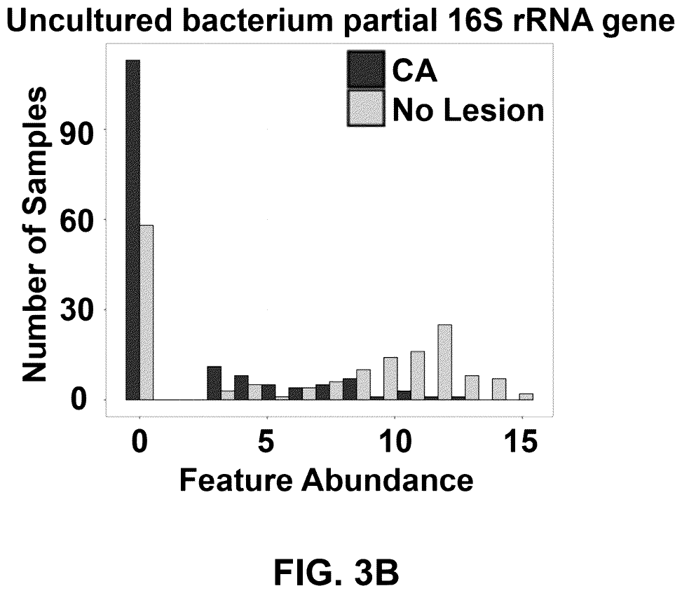

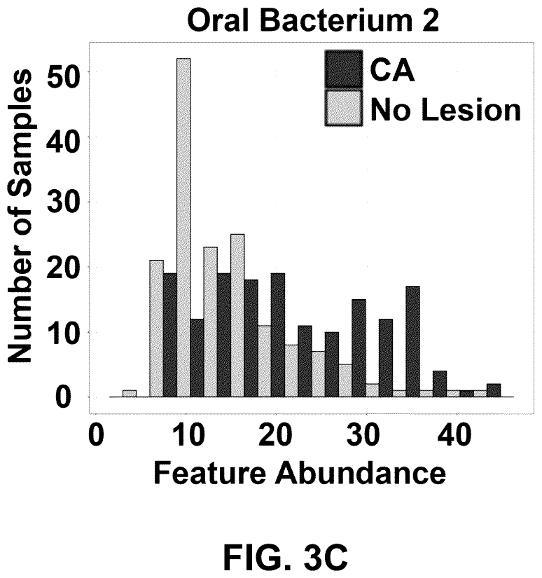

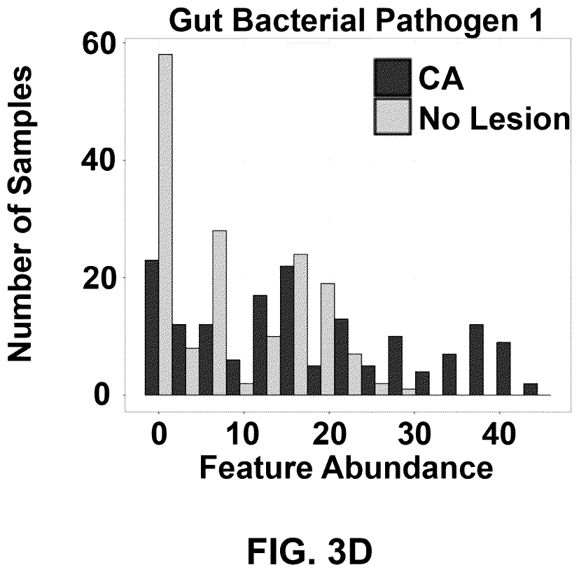

Bacterial features identified by our platform demonstrate that Fusobacteria clade-level association is unlikely to be the only example of highly specific, substrain-level differentiation of CRC-related bacteria. The ML was also able to identify bacterial signals that were expected, like Fusobacterium (FIG. 3A), but also other oral bacteria (FIG. 3C) and gut bacterial pathogens (FIG. 3D). One interesting result was that the ML identified bacterial sequence features, and therefore bacteria, rarely seen in fecal CRC-positive samples. FIG. 3B depicts a sequence feature mapping to ‘uncultured bacterium partial 16S rRNA gene’ sequence in the National Center for Biotechnology Information (NCBI) database. This feature serves as an example of a sequence feature negatively correlated with CRC-positive samples.

FIG. 3 shows CRC-specific sequence features contribute strong positive and negative correlations with CRC. A small subset of bacterial sequence features detected as part of the ML/AI algorithm for CRC screening sort with CRC and non-CRC (Normal). The y-axis counts the number of samples, the colors indicate the sample type. The sequence feature abundance is shown on the x-axis, using a log2 scale. Samples that did not have the signal are shown at ‘0’ Feature Abundance. The taxonomy/name is summarizes the best taxonomy available for that feature after mapping the NCBI database.

Features were selected and used to build classifiers for screening samples in a database-independent manner, without reference to bacterial taxonomy. However, comparison of taxonomies associated with important features can provide insight into why the ML is sorting each of the blinded samples into CA, AA and normal. Investigation in silico of the taxonomic association of a subset of features demonstrated that their utility was at least partially based on the relative abundance of the feature with respect to the CRC vs. normal samples. Furthermore, the taxonomies associated with the features frequently reflected bacterial taxa that have been consistently associated with CRC in the literature. For example, the feature in FIG. 3D mapped to a bacterial ASVs including C. difficile and Peptostreptococcus, both of which have been strongly linked to CRC. For the unknown bacterial feature associated with normal controls in FIG. 3B, the follow up investigation reinforced the original finding that it is currently unknown, even though it is found in half of the training samples. Oral Bacterium 2 in FIG. 3C contains features that map to Streptococcus, different biotypes of which have strong CRC association. Other features mapped to Bacteroidies fragilis, also a strong driver of CRC⁶. These findings are consistent with previous strain level analysis of common pathogens, which suggests that there are far more strains of familiar human-associated species than are sequenced genomes. Our ability to identify signals from completely unknown bacteria that are strongly associated with CRC, and use those features to successfully screen blinded samples is proof that the combination of strain-level resolution and database independence of our platform is a powerful and necessary feature for accurate CRC and AA screening. The finding that there are multiple features and classifiers that combine to generate signals required for accurate CRC screening is consistent with the hypothesis that multiple bacterial biofilm consortia drive colon cancer. In FIG. 3, similar to Fusobacteria, there was no single bacterial taxon or single complex feature classifier that was present only in CA, AA or normal controls.

We demonstrate identification of bacterially generated metabolites associated with CRC. Bacterial metabolites generated by the fecal microbiome have access to the local environment, but unlike bacteria, can pass into human cells, and into the bloodstream, signaling the immune system, supporting or disrupting homeostasis, and have effects far from the gut environment. Comparison of multiple bacterial metabolites in fecal samples identified a significant drop in PE DHC in both AA and CA samples as compare to normals. As with bacterial strain association, the drop was not exactly correlated with disease, but on average the difference was significant.

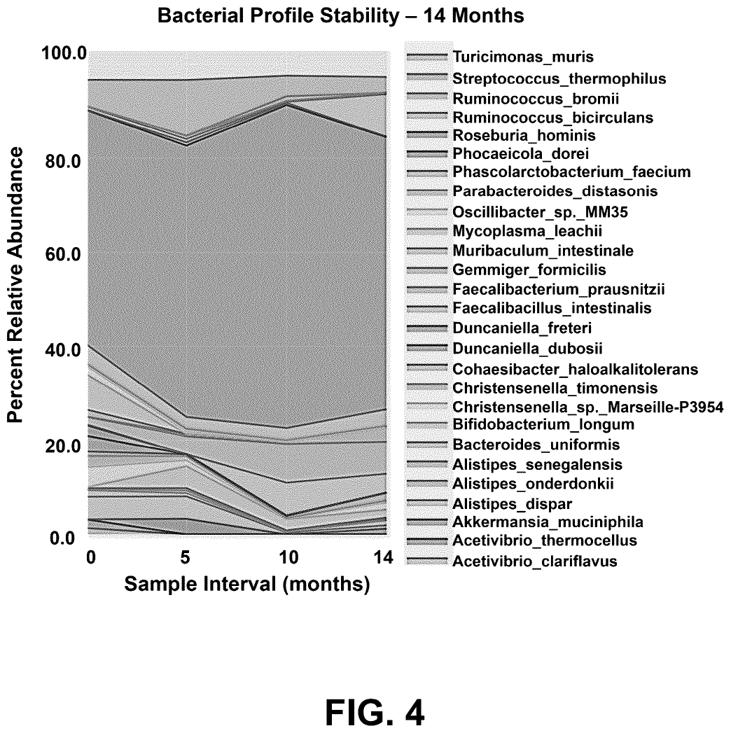

Bacterial profiles in the gut are generally stable over long time periods. It is important to understand stability and reproducibility of our platform, from sample collection through lysis, PCR, sequencing and analysis. It is reported that the microbiome in a healthy adult is generally stable, a key insight that enables measurement of meaningful changes over time. Our platform was validated and certified as a laboratory developed test. As an illustration of LDT results stability over time, longitudinal profiling of an individual using the platform was used to demonstrate that the same bacterial profile can be consistently measured over long periods in different samples from a healthy individual. As a demonstration, in FIG. 4, we followed a gut microbial profile over 14 months, where no major changes to diet, health, or lifestyle occurred. As expected, the bacterial profile was remarkably stable over time. This result indicates that our assay is able to produce consistent results over time from different samples from the same individual. Multiple samples of the same fecal material produced nearly identical results as well. The stability of the microbiome over time suggests that there is a mechanism for maintaining bacterial strains and their relative abundance that is strongly conserved that may be important for long term health. Conversely, disruptions to the stable state, as seen after antibiotic use, may have negative consequences. FIG. 4 illustrates that measurements of bacterial representation produced by our platform are stable over many months if the microbiome is stable. Conversely, this also indicates that changes to the profile are meaningful.

FIG. 4. shows a bacterial profile stable over 14 months. Fecal samples from a single adult male, age 56 at the first time point, were sampled every few months over a 14 month period. Species above 1% relative abundance are shown. There was no significant change in health status, no antibiotic use, and no major change in diet over the 14-month period.

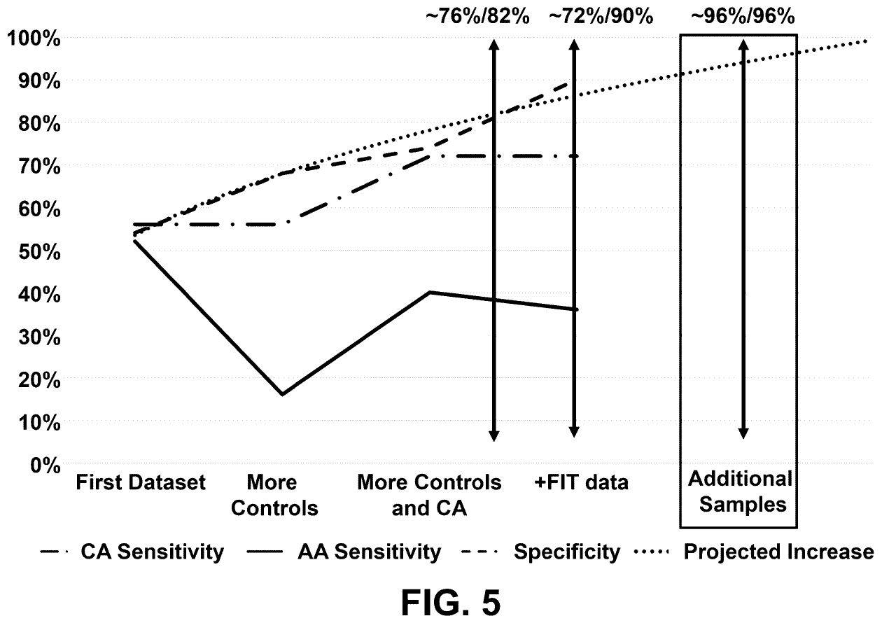

Our CRC screening preformed with the present invention is unique in that it improves with more data, which can be leveraged to increase accuracy. Trends for increased CRC screening sensitivity and specificity were confirmed with the addition of samples over four rounds of sample acquisition and ML training. In FIG. 1, the addition of cancer samples resulted in increased sensitivity for CRC (improved cancer detection, fewer false negatives), whereas increases in control samples and addition of FIT data increased specificity (fewer false positives). In FIG. 5 we use those data to project how additional samples may increase accuracy of screening to 96% or greater.

FIG. 5 shows a trendline to reach 96% or greater sensitivity and specificity. An exponential curve was used to estimate that training the ML algorithm on 50 additional CA, 65 AA, and 200 control samples can be expected to achieve 96% or better sensitivity and specificity for CRC and AA.

Results to date with a limited number of samples demonstrate that as samples were added to the training dataset, the ability to correctly screen 100 blinded samples increased rapidly. For example, the addition of 22 cancer samples resulted in a sensitivity gain of 16% for cancer detection in the blinded sample set, evidence that further addition of cancer samples should yield significant additional sensitivity gains. The addition of 78 normal controls added 20% to specificity in the blinded sample set, and the addition of FIT data added a further 16%. Interestingly, FIT results did not increase sensitivity for CRC. It appears that a low FIT score is contributing to correct differentiation of false positives, but that the bacterial content is providing high levels of CRC sensitivity. One key consideration for discussion is the AA sensitivity. There were only 35 samples available for this pilot experiment, but it was a sufficiently large sample to obtain AA accuracy similar to a currently available test for the 100 blinded samples. Based on the increase in sensitivity and specificity seen for CRC as more samples were added, it is reasonable to project similar improvements for AA as more samples are added to the training dataset in future experiments.

### Discussion of Experimental Results

This study was designed to differentiate bacterial populations associated with well-characterized CRC, AA and normal fecal samples using machine learning algorithms trained on increasing quantities of high resolution amplicon sequencing data. This amplicon includes the 16S gene, a well-studied mix of conserved and variable regions that typically enables bacterial taxonomic differentiation to the genus and sometimes the species level. The inclusion of the high variability of the ITS region in the amplicon enables differentiation of closely related strains. During training, the ML selected features with significant differences between the AA/CA and normal samples, and combined those features into classifiers. The database independent feature selection yielded features and classifiers with high discriminant power across the 16S, ITS, and 23S regions of the amplicon, each of which was derived without the need for taxonomic identification, which could also be used to screen blinded samples without the need for taxonomic identification. The classifiers were used to screen a set of 100 blinded samples, and the resulting sensitivity and specificity for CA and AA reported by Exact Sciences was similar to the results with a currently available testing product. Features selected by the ML to differentiate bacterial groups were database independent, with significance calibrated to the differences between the CRC-related cases and controls, rather than established taxonomic conventions like species or strain names, or relatively arbitrary taxonomic conventions like genomic average nucleotide identity, or housekeeping gene differences, which can help with taxonomic assignment, but which may not be important in CRC or other disease states. For example, it has long been known that Fusobacterium nucleatum (Fn) is frequently associated with CRC, but only in about half of cases. A clade-level study recently demonstrated that Fusobacterium nucleatum anamalis clade 2 (Fna C2) is enriched in CRC tumors, and other Fn strains are less likely to be associated with CRC. However, even this knowledge can have limited practical utility, because there are a limited number of Fna C2 representatives sequenced, which presents a challenge to any CRC screen using taxonomic (database dependent) information. For example, if a Fusobacterium nucleatum is identified in a fecal sample, whether it is part of the ‘Fn animalis clade 2’ or not requires a comparison of the sample Fn housekeeping genes to housekeeping genes previously used for Fn typing, which in turn requires high coverage of the entire Fn genome from that sample. Obtaining sufficient sequence of a single ˜2M base Fn genome from a sample for housekeeping gene assessment, where Fn may be present at only a few percent of the total population, requires extensive sequencing of the sample. For example, 10× coverage of a 2M base genome present at 1% relative abundance requires ˜2B bases of sequencing for that sample, followed by assembly of the genome/gene content, followed by housekeeping gene phylogenetic tree comparison and clade determination. The sequencing required for the shotgun/taxonomy-based approach is ˜80× more than the ˜10,000 reads required for obtaining a 100-fold oversampling of the ingerprint for the strain at 1% relative abundance. The time, cost and expertise required for shotgun-based taxonomic assessment is impractical for a routine assay, and an additional complication is that it has not been shown that the ‘Clade 2’ designation is the complete and correct taxonomic division for Fn-related CRC association, as it is based on housekeeping gene comparison rather than CRC disease association. In contrast, the database-independent classifiers derived from the amplicon/ML approach independently discovered C2-specific sequences in the target amplicon that were sufficient for immediate, accurate automated screening. Rather than relying on housekeeping gene comparisons to sort bacteria by function, our database independent method allows division of bacterial function with respect to the disease of interest, generating strain-level resolution, and sometimes beyond the strain level, as required for robust CRC/AA screening.

One advantage of the ML approach is that it integrates both positive and negative associations for screening at different levels of taxonomic resolution. This database-independent ML approach demonstrates that our amplicon contains sufficient information in the absence of the full genome sequence to identify both known and unknown CRC related bacterial sequence features. In addition to Fusobacteria, the ML identified CRC-associated substrain-level sequences from E. coli, Streptococcus, B. fragilis, and other gut and oral bacteria that have been reported as increased in CRC. Importantly, the ML was able to identify sequence features with strong negative correlations with CRC, that were present in higher levels in control samples. A combination of positive and negatively associated bacterial features were combined by the ML for AA and CRC screening specificity and sensitivity similar to a commercially available product, providing an explanation of why larger numbers of samples in the training dataset leads to increases in sensitivity and specificity. Strong but non-universal association of bacterial sequence features can include rare or uncharacterized pathogens and commensals that are either positively or negatively correlated with CRC, and these associations will become more significant to ML decision making at higher numbers of samples. As an example, the feature in FIG. 3B primarily sorted with non-CRC (Normal). Approximately half of samples did not have the feature. When looking across a limited number of samples at lower resolution, it is easy to understand how a background commensal like this one that is poorly characterized (not in a database) and relatively rare could be missed in database-dependent analyses, as the sequence data will not map to any known taxonomy and might be discarded. Database independence enables disease-specific discovery of bacterial sequence features, because once the sequence data become a feature as part of the classifiers, it is possible to link sequence-associated ASVs from the Intus Bio sample dataset. Even unknown bacterial features will link to one or more ASVs consisting of ˜2500 base 16S-ITS-23S amplicon sequences unique to the bacterial genomes in the training dataset. The complete ASVs enable specific sequence-level identification of the strains involved, which can guide strain isolation and characterization where appropriate. For the unknown bacterium in FIG. 3B, further investigation in silico of the taxonomic hierarchy of the sequence reinforced the original finding that it is currently unknown, even though it is found in half of the training samples, and is more common in healthy people. This finding is consistent with analysis of hospital environments, which suggests that there are far more strains of familiar human-associated pathogenic species than are characterized in NCBI². Our ability to identify completely uncharacterized bacterial sequences that are strongly associated with CRC is proof that the combination of strain-level resolution and database independence of our platform is a powerful and necessary feature for CRC screening and diagnosis.

The ML developed classifiers sufficient for accurate CRC screening that contained signals for multiple bacteria, consistent with recent findings indicating that multiple biofilm-specific bacterial consortia may be responsible for tumor instantiation. Individual bacterial features were enriched in CRC or controls, but no single bacterial signals were found in all samples. For example, we identified C. difficile as one of the consortia members⁷, but similar to Fusobacteria, about half the samples lacked the feature. Once again, in the absence of specific instructions, ML considers the simultaneous presence of multiple bacterial signals when building the classifiers.

A foundational element in the success of the ML to build accurate classifiers is the size and content of our amplicon. The relative lack of CRC-related signals in specific 16S rRNA gene variable regions may explain why previous studies using short read amplicon technology were unable to detect a robust signal for CRC-related bacteria. One interesting previous study attempted to classify CRC using data from the 16S V4 region (Baxter, et al.). The V4 region only contains about 2% of the features differentiating CRC from normal (FIG. 2). To measure the effects of the amplicon content, we tested the ability of the ML to generate features and classifiers using the published V4 dataset from Baxter, et al., to determine if the decreased data richness from the V4 amplicon could be used by the ML to correctly classify the 100 blinded sample set. Similar to the Baxter, et al., we found that the V4-dataset based ML classifiers also failed to correctly classify unknown CRC fecal samples.

The amplicon data was combined with machine learning algorithms to analyze a small, well characterized 57 sample CRC, 35 sample AA, and 256 control training sample set to identify CRC and AA-related sequence features, without reliance on taxonomy or database mapping. The features were combined into classifiers that were used to screen a blinded 100 sample test dataset provided by Exact Sciences. Classifier performance using bacterial sequences was equivalent to results from a currently available testing product on a separate, well-characterized, blinded 100 fecal sample set. Uniquely, we show that sensitivity and specificity of machine learning CRC diagnosis consistently improves with additional samples, which can be used to increase accuracy by adding more samples to the training dataset. Future work is planned to leverage the combination of high data quality, database independence, and machine learning-based feature selection to improve CRC and AA precancerous lesion detection, exceeding the benchmarks of existing CRC screening devices.

### Materials and Methods

Sample Collection: Well-characterized, anonymized AA, CA and Control samples for machine learning training were provided by Exact Sciences Corporation (Redwood City, CA, USA). Summary of diagnosis and pathology in Table NNN (supplementary file A, Table 1, ‘Training Sample Dataset 1’).

Obtaining microbiome sequence data from a biological sample may be accomplished by known methods. Releasing genetic material from bacterial cells in the biological sample typically includes subjecting the sample or bacterial cells to lysis conditions. Advantageously, proportional lysis methods such as those described in U.S. Pat. No. 10,774,322, issued Sep. 15, 2020, or U.S. Pat. No. 11,149,246, issued Oct. 19, 2021, are utilized such that the genetic material released from the bacterial cells is representative of the bacterial makeup of the sample. These patents are incorporated by reference herein for their teachings on proportional lysis methods. Preferably, though not exclusively, such proportional lysis techniques are utilized to generate retrospective and prospective patient sequence data. As otherwise described herein, unique bacterial genetic sequences may be determined from rRNA sequences, including but not limited to the 16S-ITS-23S amplicon. Such sequences and others, as well as unique sequence identification and sequencing methods, are taught in, for example, U.S. Pat. No. 10,894,990, issued Jan. 19, 2021, which is incorporated by reference herein for said teachings. PCR amplification may be accomplished by methods which eliminate primer concentration-dependent PCR amplification bias, such as those taught in US 2019/0352712, published Nov. 21, 2019, which is incorporated by reference herein for said teachings. While these methods provide advantages, any other appropriate methods may be implemented.

An additional 100 blinded, anonymized AA, CA and Control samples were provided by Exact Sciences Corporation (Redwood City, CA, USA). Diagnosis and pathology results were only known by Exact Sciences. Results were reported by Exact Sciences to Intus Bio as ‘percentage correct’, rather than on an individual sample basis, for all blinded sample experiments. Metadata for these samples is held in confidence by Exact Sciences, and is unknown to the authors as part of the continuing data collection/algorithm improvement.

Additional anonymized CA samples were provided by James Kinross. (supplementary file A, Table 2, ‘Additional CRC Training Samples) Additional anonymized normal control samples were provided by Intus Biosciences (supplementary file A, Table 3, ‘Additional Control Training Samples)

Sample Processing: DNA from fecal samples was extracted and barcoded amplicons were prepared as previously described using our Intus Biosciences Complete Kit (Intus Biosciences, Farmington CT). Briefly, samples were transferred to individual wells of a 96 well plate and subjected to lysis and purification as per manufacturer's instructions. Samples were transferred to a second provided 96-well plate containing barcoded StrainID amplicon primer sets dried down in each of 96 wells, one barcode per well. Post PCR, the samples were purified and pooled according to kit instructions for PacBio library SMRTbell preparation (cat# 100-938-900) and sequencing (Sequel IIe, PacBio). Sequencing was performed using certified Laboratory Developed Test (LDT) protocols.

ML feature selection and classifier building: Database-independent machine learning algorithms were used to generate AA/CA results for CRC screening of the 100 blinded samples test dataset.

Enclosed as a part of this provisional patent application is a document entitled “Breakthrough Platform for Testing and Discovery of Cancer, Chronic Disease Related Bacterial Profiles,” 42 pages. The material in this document is incorporated herein and should be understood in the context of this description.

As used herein, “patient” and “retrospective patient” generally mean patients having a disease state diagnosed under the appropriate medical guidelines. Sequence data from “patients” and “retrospective patients” is useful for training and validating the machine learning algorithms herein. “Patients” may be organized into cohorts having or lacking the disease state. The term “subject” may be used interchangeably with “patient.” The “patient” or “retrospective patient” may be mammalian, including human and non-human mammals. In an embodiment, the “patient” or “retrospective patient” is a human. In another embodiment, the mammalian “patient” or “retrospective patient” is bovine, equine, canine, feline, porcine, or other mammal. In further embodiments, the “patient” or “retrospective patient” may be a non-mammalian subject, including reptile, amphibian, fish, or others. As a person of skill in the art would recognize, the patient or subject may generally be any organism harboring a bacterial microbiome.

As use herein, “prospective patients” do not have a diagnosed disease state. A biological sample from a “prospective patient” may be screened using a trained machine learning model to determine whether the prospective patient has a disease state or is at risk for developing the disease state. The term “subject” may be used interchangeably with “patient.” The “patient” or “prospective patient” may be mammalian, including human and non-human mammals. In an embodiment, the “patient” or “prospective patient” is a human. In another embodiment, the mammalian “patient” or “prospective patient” is bovine, equine, canine, feline, porcine, or other mammal. In further embodiments, the “patient” or “prospective patient” may be a non-mammalian subject, including reptile, amphibian, fish, or others. As a person of skill in the art would recognize, the patient or subject may generally be any organism harboring a bacterial microbiome.

In various embodiments, it may be determined that the patient has a disease state (i.e., the characteristic of having the disease), that the patient lack a disease state (i.e., the characteristic of lacking the disease), or that the patient is at risk for developing the disease (i.e., the characteristic of being at risk for having the disease, but not yet having sufficient indicators for diagnosis under clinical guidelines). A patient “having the disease” may have any of a pre-disease stage (i.e., being at risk for disease), an early stage of a disease, an intermediate stage of a disease, or an advanced stage of a disease, depending upon the clinical guidelines for diagnosis of said disease. It should be appreciated that the present systems and methods, in various embodiments, may differentiate between these stages to determine the current disease stage of a prospective patient. As used herein, “microbiome sequence data” and variations of the term such as “microbiome nucleotide sequences” refer to bacterial genetic sequences collected from a biological sample of a patient which are from or representative of a bacterial microbiome of the patient. At least a portion of the “microbiome sequence data” is utilized to identify “features” as described herein. The at least a portion of the “microbiome sequence data” may include any useful portion thereof. In an embodiment, the “microbiome sequence data” includes or comprises the 16S-ITS-23S region (alternatively referred to as the 16S-ITS-23S amplicon). In other embodiments, regions neighboring the 16S-ITS-23S region (such as within about 2,000; 5,000; or 10,000 base pairs) may be included with or without the 16S-ITS-23S region.

As used herein, “features” identified from microbiome sequence data are portions of the microbiome sequence data which tend to be unique across various microbiome bacterial species and which may be quantified to indicate a difference in microbiome bacterial composition. The “features” need not be assigned to a certain taxonomical category and may be taxonomy and database independent. Generally, a feature will have an associated property of being negatively or positively correlated with a disease state. The property of being negatively or positively correlated with a disease state may be but is not necessarily binary, and various degrees of correlation may be implemented.

In some embodiments, “metadata” for the various patients may be useful for training and predictive capabilities of machine learning models. Such metadata may include sex, age, prior or current medication use, diet, geographical location, race, diagnoses for diseases other than the disease state for the model, or any other useful patient metadata.

As used herein, the term “biological specimen” generally encompasses any biological specimen which may contain genetic material upon which a machine learning model may be trained to correlate nucleotide sequences with a disease state. Typically, the biological specimen will contain bacterial species or genetic material from bacterial species representative of a microbiome. In some embodiments, the biological specimens are fecal samples.

In various embodiments, patient data includes “computer-readable microbiome nucleotide sequences.” It is generally contemplated that any computer-readable sequence may be utilized, as would be appreciated by a person of ordinary skill in the art. The computer-readable sequences may contain more sequence data than is necessary or desirable, in which case the methods and systems herein may truncate the sequence or extract particular regions of the sequence, as identified in the sequence data or as identified by recognition of the particular regions. Generally, any appropriate manner in which the sequence data may be accessed, loaded, and utilized is contemplated.

The term “fecal immunochemical test” refers to a test which detects the presence or absence of, or quantifies the amount of, blood present in stool from a fecal sample. In various embodiments, the “fecal immunochemical test” may encompass detection of any non-genetic material present in the stool from a fecal sample, such as bacterial markers or other biological fragments.

The systems and methods herein may correlate patient microbiome sequence data with a disease state. This correlation may include identifying the presence of the disease state, the severity of the disease state, risk for developing the disease state, or any other relevant, quantifiable aspect of the disease state of the patient. Moreover, if the patient is being treated for the disease state after an initial diagnosis, the systems and methods herein may be utilized to monitor the treatment, disease regression or progression, and/or disease remission. Additionally, if a patient having a prior disease state is now in remission of said disease state, the systems and methods may be utilized to monitor for recurrence of the disease state or recurrence of risk for re-developing the disease state.

### REFERENCES

1. Coleman, S. et al. High-resolution microbiome analysis reveals exclusionary Klebsiella species competition in preterm infants at risk for necrotizing enterocolitis. Sci. Rep. 13, 1-11 (2023).
2. Graf, J. et al. High-Resolution Differentiation of Enteric Bacteria in Premature Infant Fecal Microbiomes Using a Novel rRNA Amplicon. MBio 12, 1-18 (2021).
3. Hendricks, S. A. et al. High-Resolution Taxonomic Characterization Reveals Novel Human Microbial Strains with Potential as Risk Factors and Probiotics for Prediabetes and Type 2 Diabetes. Microorganisms 11, (2023).
4. Gehrig, J. L. et al. Finding the right fit: evaluation of short-read and long-read sequencing approaches to maximize the utility of clinical microbiome data. Microb. Genomics 8, (2022).
5. Dejea, C. M. et al. Microbiota organization is a distinct feature of proximal colorectal cancers. Proc. Natl. Acad. Sci. U.S.A. 111, 18321-18326 (2014).
6. El Tekle, G. & Garrett, W. S. Bacteria in cancer initiation, promotion and progression. Nat. Rev. Cancer 23, 600-618 (2023).
7. Drewes, J. L. et al. Human Colon Cancer-Derived Clostridioides difficile Strains Drive Colonic Tumorigenesis in Mice. Cancer Discov. 12, 1873-1885 (2022).
8. Tjalsma, H., Boleij, A., Marchesi, J. R. & Dutilh, B. E. A bacterial driver-passenger model for colorectal cancer: Beyond the usual suspects. Nat. Rev. Microbiol. 10, 575-582 (2012).
9. Baxter, N. T., Ruffin, M. T., Rogers, M. A. M. & Schloss, P. D. Microbiota-based model improves the sensitivity of fecal immunochemical test for detecting colonic lesions. Genome Med. 8, 1-10 (2016).
10. Hanna, M., Dey, N. & Grady, W. M. Emerging Tests for Noninvasive Colorectal Cancer Screening. Clin. Gastroenterol. Hepatol. 21, 604-616 (2023).
11. Betge, J. & Ebert, M. P. Unveiling the culprit: the fusobacterium lineage that populates colorectal cancer. Signal Transduct. Target. Ther. 9, 8-9 (2024).
12. Zepeda-Rivera, M. et al. A distinct Fusobacterium nucleatum clade dominates the colorectal cancer niche. Nat. 2024 1-9 (2024) doi:10.1038/s41586-024-07182-w.
13. Oren, A. & Garrity, G. M. Valid publication of the names of forty-two phyla of prokaryotes. Int. J. Syst. Evol. Microbiol. 71, (2021).
14. Qiao, N. et al. After the storm-Perspectives on the taxonomy of Lactobacilluseae. JDS Commun. 3, 222-227 (2022).
15. Ferraz Helene, L. C., Klepa, M. S. & Hungria, M. New Insights into the Taxonomy of Bacteria in the Genomic Era and a Case Study with Rhizobia. Int. J. Microbiol. 2022, (2022).
16. Blackwell, G. A. et al. Exploring bacterial diversity via a curated and searchable snapshot of archived DNA sequences. PLoS Biol. 19, (2021).
17. Sayers, E. W. et al. Database resources of the national center for biotechnology information. Nucleic Acids Res. 50, D20-D26 (2022).
18. Liu, Y. et al. Peptostreptococcus anaerobius mediates anti-PD1 therapy resistance and exacerbates colorectal cancer via myeloid-derived suppressor cells in mice. Nature Microbiology (Springer US, 2024). doi:10.1038/s41564-024-01695-w.
19. Boleij, A., Van Gelder, M. M. H. J., Swinkels, D. W. & Tjalsma, H. Clinical importance of streptococcus gallolyticus infection among colorectal cancer patients: Systematic review and meta-analysis. Clin. Infect. Dis. 53, 870-878 (2011).
20. Ahmed S, A., Rand R, H. & Fatimah Abu, B. The association of Streptococcus bovis/gallolyticus with colorectal tumors: The nature and the underlying mechanisms of its etiological role. J. Exp. Clin. Cancer Res. 30, 1-13 (2011).
21. O'Brien, C. L., Allison, G. E., Grimpen, F. & Pavli, P. Impact of Colonoscopy Bowel Preparation on Intestinal Microbiota. PLOS One 8, 1-10 (2013).
22. Zhou, X. et al. Longitudinal profiling of the microbiome at four body sites reveals core stability and individualized dynamics during health and disease. Cell Host Microbe 1-21 (2024) doi:10.1016/j. chom.2024.02.012.
23. Aprile, F. et al. Microbiota alterations in precancerous colon lesions: A systematic review. Cancers (Basel). 13, 1-14 (2021).
24. Morrison, A. G., Sarkar, S., Umar, S., Lee, S. T. M. & Thomas, S. M. The Contribution of the Human Oral Microbiome to Oral Disease: A Review. Microorganisms 11, 1-17 (2023).
25. Hong, B. Y., Driscoll, M., Gratalo, D., Jarvie, T. & Weinstock, G. M. Improved DNA Extraction and Amplification Strategy for 16S rRNA Gene Amplicon-Based Microbiome Studies. Int. J. Mol. Sci. 25, (2024).

### INCORPORATON BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the present invention, where the term comprises is used with respect to the recited components or steps of the platforms or methods, it is also contemplated that the platforms and methods consist essentially of, or consist of, the recited components or steps. Furthermore, the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

All percentages and ratios used herein, unless otherwise indicated, are by weight.

What is claimed is:

1. A method for diagnosing or predicting the development of a disease state from microbiome sequence data from a prospective patient, comprising the following steps:
a. collecting biological specimens and metadata from a patient cohort having the disease state and from a patient cohort lacking the disease state;
b. generating microbiome sequence data from the biological specimens;
c. processing the microbiome sequence data to generate features having a quantified relevance to the disease state for each patient;
d. associating metadata with the generated features for each patient;
e. selecting a subset of the features to generate a reduced feature set;
f. training a machine learning algorithm on the reduced feature set to create a classification model that classifies the patient status as having the disease state, lacking the disease state, or being at risk for developing the disease state;
g. obtaining microbiome sequence data and metadata from a prospective patient;
h. quantifying the features in the reduced feature set from the microbiome sequence data of the prospective patient; and
i. applying the classification model to the quantified features in the reduced feature set from the prospective patient to determine whether the prospective patient has or lacks the disease state, or are is risk for developing the disease state.

2. The method of claim 1, wherein the features can be matched and compared across patients.

3. The method of claim 2, further comprising applying data transformations to calibrate, normalize, or quantize the features for comparison across patients.

4. The method of claim 1, where the microbiome sequence data is the 16S rRNA gene and flanking upstream and downstream genomic regions, in part or in whole.

5. The method of claim 1, where the microbiome sequence data begins in or upstream of the 16S rRNA gene and extends past the end of the 16S rRNA gene as a contiguous amplicon sequence.

6. The method of claim 1, wherein the microbiome sequence data comprises one or more of 16S, ITS, and 23S sequences.

7. The method of claim 6, wherein the microbiome sequence data comprises the 16S-ITS-23S amplicon.

8. The method of claim 1, where the quantified relevance to the disease state of each feature is determined by (i) grouping the reads across samples into Operational Taxonomic Units (OTUs) or Amplicon Sequence Variants (ASVs),
(ii) creating a representative sequence for each OTU or ASV, and
(iii) counting the number of reads matching each OTU or ASV representative in each sample.

9. The method of claim 1, where the quantified relevance to the disease state of each feature is defined to be the number of occurrences of the feature in the microbiome sequence data.

10. The method of claim 1, wherein the length of each feature is approximately 5 to 100 nucleotides.

11. The method of claim 1 in which the biological specimens are fecal samples, blood samples, CSF samples, urine samples, saliva samples, other internal or external bodily fluids, skin swabs, gum swabs, vaginal swabs, or swabs of specific internal or external anatomical features.

12. The method of claim 1, further comprising:
obtaining fecal immunochemical test data from one or more of the patient cohort having the disease state and the patient cohort lacking the disease state; and
training the machine learning algorithm on the reduced feature set and the fecal immunochemical test data to create the classification model.

13. The method of claim 12, further comprising:
obtaining fecal immunochemical test data from the prospective patient; and
applying the classification model to the quantified features in the reduced feature set from the prospective patient and the fecal immunochemical test data from the prospective patient to determine whether the prospective patient has or lack the disease state, or are is risk for developing the disease state.

14. The method of claim 1, in which the disease state is a neurodegenerative disease, an Alzheimer's Disease, Parkinson's Disease, Amyotrophic Lateral Sclerosis (ALS), Multiple Sclerosis (MS), Lewy Body Dementia, Frontotemporal Dementia, Spinocerebellar Ataxia, autoimmune disease, Celiac Disease, Crohn's Disease, Ulcerative Colitis, Inflammatory Bowel Disease (IBD), Rheumatoid Arthritis, Type 1 Diabetes, Hashimoto's Thyroiditis, Graves' Disease, Psoriasis, Sjögren's Syndrome, Systemic Lupus Erythematosus (SLE), Myasthenia Gravis, Vasculitis, Pemphigus Vulgaris, Dermatomyositis, Guillain-Barré Syndrome, digestive disorder, Diverticulitis, Pancreatitis, Irritable Bowel Syndrome (IBS), Gastroesophageal Reflux Disease (GERD), Peptic Ulcer Disease, Non-Alcoholic Fatty Liver Disease (NAFLD), metabolic disorders, Type 2 Diabetes, Obesity, Hyperthyroidism, Hypothyroidism, cardiovascular disease, Coronary Artery Disease, Hypertension (High Blood Pressure), Congestive Heart Failure, Stroke, Atherosclerosis, Renal (Kidney) disease, Chronic Kidney Disease (CKD), Polycystic Kidney Disease, Nephrotic Syndrome, Cancer, Lung Cancer, Breast Cancer, Prostate Cancer, Colon Cancer, Colorectal Cancer, Early Onset Colorectal Cancer, Leukemia, Lymphoma, Pancreatic Cancer, Ovarian Cancer, Melanoma, Bladder Cancer, Liver Cancer, Kidney (renal cell and renal pelvis) Cancer, mental health disorder, Depression, Anxiety Disorders, Bipolar Disorder, Schizophrenia, Obsessive-Compulsive Disorder (OCD), Post-Traumatic Stress Disorder (PTSD), substance use disorder, Alcohol Use Disorder, Opioid Use Disorder, Nicotine Dependence, Chronic Obstructive Pulmonary Disease (COPD), Asthma, Fibromyalgia, Gout, Osteoarthritis, and Osteoporosis.

15. A method of training a machine learning algorithm to correlate patient microbiome sequence data with a disease state:
obtaining sequence data for a first plurality of patients having a diagnosed disease state and for a second plurality of control patients lacking the disease state, wherein the sequence data of the first and second pluralities of patients comprises respective computer-readable microbiome nucleotide sequences from biological samples collected from the respective patients;
identifying sequence features from the microbiome nucleotide sequences which correlate positively or negatively with the disease state;
generating machine learning training data comprising:
i) at least a subset of the identified sequence features,
ii) for each of the identified sequence features, their property of corellating positively or negatively with the disease state, and
iii) retrospective patient data comprising computer-readable microbiome nucleotide sequences from biological samples collected from a plurality of retrospective patients having the disease state and/or a plurality of retrospective patients lacking the disease state; and
training the machine learning algorithm with the machine learning training data to predict the presence or absence of the disease state in the retrospective patient data.

16. The method of claim 15, wherein training the machine learning algorithm produces a model capable of predicting the presence or absence of the disease state in prospective patients having no known disease state.

17. The method of claim 15, wherein the method includes no taxonomic identification of bacterial strains in the microbiome nucleotide sequences.

18. The method of claim 15, wherein the microbiome nucleotide sequences comprise bacterial nucleotide sequences.

19. The method of claim 18, wherein the bacterial nucleotide sequences comprise one or more of 16S, ITS, and 23S sequences.

20. The method of claim 19, wherein the bacterial nucleotode sequences comprise the 16S-ITS-23S amplicon.

21. The method of claim 20, wherein the training data includes no taxonomic identification bacterial strains from the 16S-ITS-23S amplicons.

22. The method of claim 15, wherein the sequence data and retrospective patient data are proportional to the bacterial populations in the underlying biological samples.

23. The method of claim 15, wherein the machine learning training data further comprises retrospective patient data comprising computer-readable microbiome nucleotide sequences from biological samples collected from a plurality of retrospective patients lacking the disease state.

24. The method of claim 15, in which the disease state is a neurodegenerative disease, Alzheimer's Disease, Parkinson's Disease, Amyotrophic Lateral Sclerosis (ALS), Multiple Sclerosis (MS), Lewy Body Dementia, Frontotemporal Dementia, Spinocerebellar Ataxia, autoimmune disease, Celiac Disease, Crohn's Disease, Ulcerative Colitis, Inflammatory Bowel Disease (IBD), Rheumatoid Arthritis, Type 1 Diabetes, Hashimoto's Thyroiditis, Graves' Disease, Psoriasis, Sjögren's Syndrome, Systemic Lupus Erythematosus (SLE), Myasthenia Gravis, Vasculitis, Pemphigus Vulgaris, Dermatomyositis, Guillain-Barré Syndrome, digestive disorder, Diverticulitis, Pancreatitis, Irritable Bowel Syndrome (IBS), Gastroesophageal Reflux Disease (GERD), Peptic Ulcer Disease, Non-Alcoholic Fatty Liver Disease (NAFLD), metabolic disorders, Type 2 Diabetes, Obesity, Hyperthyroidism, Hypothyroidism, cardiovascular disease, Coronary Artery Disease, Hypertension (High Blood Pressure), Congestive Heart Failure, Stroke, Atherosclerosis, Renal (Kidney) disease, Chronic Kidney Disease (CKD), Polycystic Kidney Disease, Nephrotic Syndrome, Cancer, Lung Cancer, Breast Cancer, Prostate Cancer, Colon Cancer, Colorectal Cancer, Early Onset Colorectal Cancer, Leukemia, Lymphoma, Pancreatic Cancer, Ovarian Cancer, Melanoma, Bladder Cancer, Liver Cancer, Kidney (renal cell and renal pelvis) Cancer, mental health disorder, Depression, Anxiety Disorders, Bipolar Disorder, Schizophrenia, Obsessive-Compulsive Disorder (OCD), Post-Traumatic Stress Disorder (PTSD), substance use disorder, Alcohol Use Disorder, Opioid Use Disorder, Nicotine Dependence, Chronic Obstructive Pulmonary Disease (COPD), Asthma, Fibromyalgia, Gout, Osteoarthritis, and Osteoporosis.

25. The method of claim 15, wherein the machine learning training data further comprises:
iv) fecal immunochemistry test collected from at least one of the plurality of retrospective patients having or lacking the disease state.

26. The method of claim 15, wherein the machine learning training data further comprises metadata from at least one of the plurality of retrospective patients having or lacking the disease state.

27. A system comprising;
a computing device operable to execute computer-readable instructions, the computer-readable instructions being configured to perform the steps of:
obtaining sequence data for a first plurality of patients having a diagnosed disease state and for a second plurality of control patients lacking the disease state, wherein the sequence data of the first and second pluralities of patients comprises respective computer-readable microbiome nucleotide sequences from biological samples collected from the respective patients;
identifying sequence features from the microbiome nucleotide sequences which correlate positively or negatively with the disease state ;
generating machine learning training data comprising:
i) at least a subset of the identified sequence features,
ii) for each of the identified sequence features, their property of corellating positively or negatively with the disease state, and
iii) retrospective patient data comprising computer-readable microbiome nucleotide sequences from biological samples collected from a plurality of retrospective patients having the disease state and/or a plurality of retrospective patients lacking the disease state; and
training a machine learning algorithm with the machine learning training data to predict the presence or absence of the disease state in the retrospective patient data.

28. A kit for diagnosing or predicting the development of a disease state from microbiome sequence data from a prospective patient, comprising:
a sample collector for obtaining biological specimens from a prospective patient and instructions for obtaining the biological specimens; wherein the collected biological specimens are useful for one or more of:
a. generating microbiome sequence data from the biological specimens;
b. processing the microbiome sequence data to generate features having a quantified relevance to the disease state for each patient;
c. associating metadata with the generated features for each patient;
d. selecting a subset of the features to generate a reduced feature set;
e. training a machine learning algorithm on the reduced feature set to create a classification model that classifies the patient status as having the disease state, lacking the disease state, or being at risk for developing the disease state;
f. obtaining microbiome sequence data and metadata from a prospective patient;
g. quantifying the features in the reduced feature set from the microbiome sequence data of the prospective patient; and
h. applying the classification model to the quantified features in the reduced feature set from the prospective patient to determine whether the prospective patients has or lacks the disease state, or are is risk for developing the disease state.
